(19) United States
(12) Patent Application Publication
(10) Pub. No.: US 2012/0135873 A1
(43) Pub. Date: May 31, 2012

(54) GENOME DETERMINATION ASSAY

(76) Inventor: Noga PORAT, Tel Aviv (IL)

(21) Appl. No.: 12/021,307

(22) Filed: Jan. 29, 2008

Related U.S. Application Data
(60) Provisional application No. 60/886,952, filed on Jan. 29, 2007.

(51) Int. Cl. C12Q 1/68; C12Q 1/70; C40B 30/04

(52) U.S. Cl. 506/9; 435/6.11; 435/5

(57) ABSTRACT

The present invention relates to a genome determination assay and method for detecting and cleaving a target non-amplified genomic nucleic acid sequence at two or more genomic nucleic acid sequence sites simultaneously, using a mismatch repair enzyme selected from the group consisting of TDG and Mut Y. The method may be used to detect a species under non-lab conditions and identify a DNA sequence utilizing the specificity of the base excision repair (BER) system enzymes. It may also be used to cleave a specific genomic sequence of choice. Currently, genomic DNA is only cleaved with restriction enzymes at restriction sites. As one example, chicken genome specific sequences are utilized to determine chick gender, without the use of standard assays, such as PCR or Fluorescent In Situ Hybridization (FISH). TDG, a Base Excision Repair (BER) enzyme that restores T/G mismatches to C/G at sites of 5-methylcytosine deamination, is used to detect, bind and function on a primer hybridized to genomic DNA template. The primer sequence may contain a T to mismatch a G in the target genomic DNA sequence or, symmetrically, the mismatch may be reversed so that the primer sequence may contain a G to mismatch a Tin the target genomic DNA sequence, and the T/G is cleaved with high fidelity.

### FIELD AND BACKGROUND OF THE INVENTION

The present invention relates to a genome determination assay to detect a species under non-lab conditions, and a method to determine the presence or absence and nature of a target DNA sequence, utilizing the specificity of the base excision repair (BER) system enzymes. Specifically, an assay to determine chick gender is provided.

Every living organism has its own unique DNA sequences. These sequences, now being revealed by genome sequencing projects, provide a useful tool for identifying these organisms. This is useful in Biodefense and warfare situations, or Point-of-Care settings to detect a threat of a bacterium or virus that may serve as a pathogenic agent causing disease. It also has applications in food production, as in most livestock species one sex has significantly greater market value than the other. With chickens raised for egg production, there is a much greater demand for female chicks. The assay may be used to determine chick gender in ovo in the hatchery.

Development of a quick, accurate, inexpensive automated method to sex segregate eggs before hatching would significantly increase profitability of the global chicken industry. Morally, there is the disturbing fact that egg production systems currently provide for disposal of male chicks shortly after hatching because egg-laying strains are not suitable for meat production. This is usually done by carbon dioxide gassing, decapitation, neck-breaking or suffocation (1). Approximately two billion male chicks are killed annually (2). A method which would sort eggs early in their incubation period is required, so that male chick eggs are disposed of before hatching. Thus, the benefits of automated sexing to determine the gender of chicks while still in the egg include 1) better feed and processing efficiencies; 2) reduced incubation space requirements; 3) speedier hatchery processing; and 4) reduced animal welfare concerns associated with discarding male chicks in the layer egg industry.

The sex chromosomes in birds are the reverse of mammals. Male birds are homogametic, having two Z chromosomes (ZZ), while females are heterogametic, having one Z and one W chromosome (WZ) (3,4). As a result, the sex of the offspring is determined by the genetic content of the egg rather than the sperm (5,6).

Different techniques have been suggested, using DNA, to determine chick gender. PCR and restriction enzyme analysis have been developed based on the sex-linked marker on the W chromosome, the CHD gene (7). W and Z karyotyping have been suggested (8). These may be the preferred methods for very expensive individual birds, but it is otherwise uneconomical.

Determining DNA sequences in point-of-care settings is a major challenge. To detect DNA in a very short time, such as in a doctor's office and at first responder sites such as battlefields or in other cases of Biodefense, requires straightforward, fast, low cost and high accuracy (a high signal to noise ratio) assays. Currently this requires use of amplification methodologies, well known to those skilled in the art, such as polymerase chain reaction (PCR), fluorescent in situ hybridization (FISH), ligase chain reaction (LCR), nucleic acid system-based amplification (NASBA), and cycling probe technology (CPT). Polymerase chain reaction (PCR) is currently the main method used in most biological applications for amplifying, ‘singling out’, or detecting a specific gene. However, the drawback to PCR in Genomic DNA detection in non-lab settings is the exponential amplification factor in PCR that allows amplification of contaminants, which are abundant in these types of settings. Similarly, post-synthetic, epigenetic modifications of genomic DNA, such as methylation, are lost in the PCR process. Existing methods such as Bisulfite PCR and cleavage by methylation-specific restriction enzymes, each have their disadvantages. Bisulfite PCR consists of numerous steps, which may lead to inconclusive results and restriction enzymes cleavage leads to excess noise. An approach for isolating a specific genomic sequence without PCR amplification would be advantageous in allowing detection of methylation patterns directly from genomic DNA.

Typically, genomic DNA is cleaved at restriction sites by restriction enzymes. Cleavage of genomic DNA at a specific genomic sequence of choice has not previously been shown, nor has insertion of a point mutation in order to cleave genomic DNA at the selected specific location for the purpose of detecting a sequence in the genomic DNA. By “a specific genomic sequence of choice” is meant a selected sequence that may be cleaved independent of the location of a common and known restriction site.

U.S. Pat. No. 5,656,430 to Lu-Change et al. discloses a method for identifying single base pair mismatches in nucleic acids using mismatch endonucleases and a set of labeled oligonucleotide probes which hybridize to mismatch sequences in the target nucleic acid such that a detectable enzyme-nucleic acid-probe complex forms or labeled cleaved fragments form when a base pair mismatch is present. The object of U.S. Pat. No. 5,656,430 is to identify a single base pair mismatch, and the method is limited to the detection of the point mutation (i.e., mismatch) to be detected. In contrast, the objective of the present invention is to identify the whole target genomic DNA sequence, and the method includes the step of inserting a point mutation at a desired location. U.S. Pat. No. 5,656,430 does not disclose detecting and cleaving single base pair mismatches on genomic DNA and the use of repetitive sequences.

U.S. Pat. No. 5,683,877 to Chirikjian, et al. discloses a method for detecting point mutations in nucleic acid sequences and method for determining the repair index for a mismatched or damaged oligonucleotide probe. However, neither U.S. Pat. No. 5,656,430 nor U.S. Pat. No. 5,683,877 disclose a method for detecting and cleaving a target non-amplified genomic nucleic acid sequence at two or more genomic nucleic acid sequence sites simultaneously, using a mismatch repair enzyme selected from the group consisting of TDG and Mut Y (9,10). Nor do U.S. Pat. Nos. 5,656,430 and 5,683,877 disclose the use of repetitive sequences to replace PCR and/or repeating the method steps numerous times. Cleaving the target non-amplified genomic nucleic acid sequence at two locations has not previously been shown.

Presently, many assays that attempt to replace PCR in detecting specific DNA in complex conditions are nanostructure-based, but have only been tested on synthetic primers. Very few have proven effective for detecting genomic DNA (11). Third Wave Technologies, Inc.'s clevase assay is an example of an assay that directly detects genomic DNA. However, the cleavase assay comprises more than one step, is time-consuming and expensive. To determine DNA sequences in point-of-care settings would require finding alternatives to time consuming, costly, and laborious laboratory assays such as the cleavase assay, PCR or FISH.

In light of the foregoing, there is a need for an accurate, one step, efficient and inexpensive method to directly detect genomic DNA from unamplified DNA source molecules. Such a method would save time, require minimal equipment, and be useful in point-of-care settings. In addition, the development of a quick, inexpensive, automated method to identify a species via its unique genome sequences in non-lab settings and more particularly, to sex segregate eggs before hatching, is highly desirable.

### SUMMARY OF THE INVENTION

The assay of the invention may be used to identify a species via its unique genome sequences in non-lab settings, utilizing the specificity of the base excision repair (BER) system enzymes and without resort to costly, time consuming and laborious amplification methodologies such as PCR or FISH to increase the number of copies of the nucleic acid sequence of interest. For instance, using the chicken genome as a DNA model, chicken genome repetitive sequences are used to determine gender. The assay of the invention thus has utility in the poultry industry to distinguish between female and male chicks in ovo in the hatchery.

The assay of the invention to directly detect non-amplified genomic DNA relies upon the ability of the base repair enzyme TDG to recognize and cleave an intact T, mismatched with G, solely in double-stranded DNA. TDG restores T/G mismatches to C/G at sites of methylcytosine deamination. TDG is shown to recognize a short probe hybridized to a target non-amplified genomic DNA sequence, wherein at one point in the sequence of the primer, C is replaced with T to mismatch G in the target sequence, and TDG is able to cleave the T in the T/G correctly. This permits differentiating between two genomes, and specifically between female and male chicken genomes.

To date, repair enzymes have been limited to use in SNP detection assays (12), and only DNA Polymerase has been shown to recognize and bind to a primer on genomic DNA, in vitro. The invention demonstrates that, similar to DNA Polymerase, TDG and Mut Y can recognize, bind and function on a primer hybridized to a genomic DNA template. If the probe sequence included a T to mismatch a G in the target genomic DNA sequence, or a G to mismatch a T in the target genomic DNA sequence, then TDG was able to detect and cleave the T in the T/G or G/T in the probe/genomic DNA hybrid with high fidelity. If the probe sequence included an A to mismatch a G in the target genomic DNA sequence, or a G to mismatch an A in the target genomic DNA sequence, then Mut Y can detect and cleave the A/G or G/A in the probe/genomic DNA hybrid with high fidelity. Thus, TDG, and other base excision repair enzymes such as Mut Y, are important tools in molecular biology, useful in ways other than their standard use in SNP detection. TDG and Mut Y can be used as tools much like DNA Polymerase and restriction enzymes.

The assay of the invention permits cleaving of a specific genomic sequence of choice. As discussed above, TDG detects a short probe hybridized to genomic DNA template and cleaves the thymine in the probe sequence that mismatches the guanine in the genomic sequence. From symmetrical considerations, however, if the mismatch is reversed, so that the guanine G is in the probe and the thymine T is in the genomic sequence, the target single-stranded genomic DNA may be cleaved at that point in its sequence. If this is done in two locations on the same strand simultaneously, for example, but not limited to, a distance of 200 nt apart, cleavage of a specific genome sequence is achieved. A scheme of the assay is presented in FIGS. 7A-7D and described in Example VII below. Specific genome sequence isolation may allow a variety of future applications. Cleavage of a specific genome sequence has currently only been shown by the engineering of zinc fingers linked to a nuclease (13, 14).

It is therefore an object of the present invention to provide a method for detecting and cleaving a target non-amplified genomic nucleic acid sequence at two or more genomic nucleic acid sequence sites simultaneously, using a mismatch repair enzyme selected from the group consisting of TDG and Mut Y, comprising, for each genomic nucleic acid sequence site to be detected and cleaved, the steps of (a) denaturing the target genomic nucleic acid base sequence; (b) preparing at least one labeled probe comprising a single-stranded nucleic acid having a target-specific genomic sequence portion complementary to a portion of the target genomic nucleic acid base sequence and a non-target-specific sequence that mismatches the target genomic nucleic acid base sequence; (c) hybridizing the at least one labeled probe with the denatured target genomic nucleic acid base sequence at two or more sites on the target genomic nucleic acid base sequence to form a probe-target genomic nucleic acid base sequence hybrid including at least one mismatch; (d) exposing the hybrid of step (c) to at least one repair enzyme selected from the group consisting of TDG and Mut Y, wherein the repair enzyme detects and cleaves the at least one mismatch to produce a cleaved nucleic acid base sequence; and (e) detecting the cleaved nucleic acid base sequence of step (d).

According to a further feature of the present invention, the probe-target genomic nucleic acid base sequence hybrid of step (c) comprises a T/G mismatch wherein a T is synthesized in the probe sequence to mismatch a G located in the genomic nucleic acid base sequence.

According to a further feature of the present invention, the probe-target genomic nucleic acid base sequence hybrid of step (c) comprises a T/G mismatch wherein a G is synthesized in the probe sequence to mismatch a T located in the genomic nucleic acid base sequence.

According to another feature of the present invention, the probe-target genomic nucleic acid base sequence hybrid of step (c) comprises an A/G mismatch wherein an A is synthesized in the probe sequence to mismatch a G located in the genomic nucleic acid base sequence.

According to yet another feature of the present invention, the probe-target genomic nucleic acid base sequence hybrid of step (c) comprises an A/G mismatch wherein a G is synthesized in the probe sequence to mismatch an A located in the genomic nucleic acid base sequence.

According to still another feature of the present invention, the method comprises, after step (e), the step of purifying the cleaved nucleic acid base sequence.

According to a further feature of the present invention, the probe is labeled with at least one molecule selected from the group consisting of a fluorescent label and black hole quencher, a fluorescent nucleotide, a fluorescent dye, biotin, derivative of biotin, radioactive molecule, fluorescent molecule, antibody, antibody fragment, hapten, carbohydrate, phosphorescent moiety, luminescent moiety, electrochemiluminescent moiety, chromatic moiety, nanostructure particle, and moiety having a detectable electron spin resonance, electrical capacitance, dielectric constant and electrical conductivity.

According to a further feature of the present invention, detection of the labeled probe in step (e) is by a method selected from the group consisting of PAGE, agarose gel, fluorescence reader detection system, sequencing, ELISA, mass spectrometry, fluorometry, hybridization, microarray, and Southern Blot.

According to another feature of the present invention, the cleaved nucleic acid base sequence of step (d) is labeled by hybridization to a biotin or fluorescent probe. The labeled, cleaved nucleic acid base sequence of step (d) may then be detected and purified.

According to a further feature of the present invention, the target genomic nucleic acid base sequence is obtained from a source selected from the group consisting of a bacterium, fungus, virus, protozoan, plant, animal and human.

According to a further feature of the present invention, the target genomic nucleic acid base sequence is obtained from a chicken.

According to yet another feature of the present invention, there is provided a kit for determining the gender of a bird or egg of unknown gender, comprising (a) a sample genomic nucleic acid base sequence from a bird or target egg of interest of unknown gender; (b) at least one hybridization probe comprising a single-stranded nucleic acid having a target-specific genomic sequence portion complementary to a portion of the target genomic nucleic acid base sequence and a non-target-specific sequence that mismatches the target genomic nucleic acid base sequence or said at least one probe joined to a label; (c) at least one repair enzyme selected from the group consisting of TDG and Mut Y; and (d) instructions for determining the gender of the bird or egg.

### BRIEF DESCRIPTION OF THE FIGURES

The invention is herein described, by way of example only, with reference to the accompanying drawings, wherein:

FIG. 1A is a schematic showing an especially-designed probe hybridized to the target genomic DNA repetitive sequence.

FIG. 1B is a schematic showing the probe/genomic DNA hybrid recognized by the mismatch repair enzyme TDG.

FIG. 1C is a schematic showing the probe cleaved by TDG after having identified the T/G, where FRET is separated and a resulting signal is then formed and analyzed in accordance with the method of the present invention;

FIG. 2A shows a PAGE gel demonstrating a 152 bp PCR product amplified from the CHD gene using the method of the present invention.

FIG. 2B shows the results of a PAGE gel showing a 164 bp PCR product amplified from the CHD gene hybridized to a 33 bp probe.

FIG. 3 shows the results of a PAGE gel showing an 18 bp FRET probe/target hybrid comprising either a T/G mismatch or a T/A match.

FIG. 4A shows the results of a laser detection system for an 18 bp FRET probe/target hybrid with a T/G mismatch, at 520 nm.

FIG. 4B shows the results of a laser detection system for an 18 bp FRET probe/target hybrid with a T/A mismatch, at 520 nm.

FIG. 5 shows the results of a PAGE gel showing non-amplified genomic DNA hybridized to a 30 bp probe.

FIG. 6A shows the results of a PAGE gel showing non-amplified genomic DNA hybridized to 33 bp probe, 15 minutes incubation.

FIG. 6B shows the results of a PAGE gel showing non-amplified genomic DNA hybridized to 33 bp probe, one hour incubation.

FIG. 6C shows the results of a PAGE gel showing non-amplified genomic DNA hybridized to 33 bp probe, 3 hours incubation.

FIGS. 7A-7D illustrates a scheme for ‘Specific Genome Sequence Isolation’ (SGSI). In FIG. 7A, two specially-designed probes are hybridized to the target genomic DNA sequences. In FIG. 7B, the probe/genomic DNA hybrids are detected by the mismatch repair enzyme TDG. In FIG. 7C, the specific genomic DNA sequence is cleaved by TDG having identified the thymines in the target mismatches. In FIG. 7D, a biotinilated probe is attached to the cleaved sequence and subsequently isolated through avidin-magnet purification.

### DESCRIPTION OF THE PREFERRED EMBODIMENTS

The present invention is directed to a genome detection assay and a method for detecting and cleaving a target non-amplified genomic nucleic acid sequence at two or more genomic nucleic acid sequence sites simultaneously, using a mismatch repair enzyme selected from the group consisting of TDG and Mut Y, which assay and method is suitable for non-lab settings. In accordance with the method of the invention, it is possible to construct a probe that is hybridized to genomic DNA. Use of BER enzymes that work on the probe in accordance with their specific functions creates a signal to be detected. The principles and operation of the methods according to the present invention may be better understood with reference to the drawings and the accompanying description.

The assay is suitable for detecting an entire genome or “whole” sequence rather than one base mismatch or a single gene. The method of the invention does not require PCR as it employs repetitive sequences to achieve amplification. The method includes no washing steps, no laborious tasks, and most importantly, it takes only minutes until detection. The assay is a solution-based assay that is straightforward, fast, low cost and highly accurate, adapted to meet point-of-care standards, with no specialized or sophisticated equipment required and no lab hassle. The invention makes use of the base excision repair (BER) system that allows the identification and excision of aberrant bases.

The method for detecting and cleaving a target non-amplified genomic nucleic acid sequence at two or more genomic nucleic acid sequence sites simultaneously, using a mismatch repair enzyme selected from the group consisting of TDG and Mut Y, comprises, for each genomic nucleic acid sequence site to be detected and cleaved, the steps of:
(a) denaturing the target genomic nucleic acid base sequence;
(b) preparing at least one labeled probe comprising a single-stranded nucleic acid having a target-specific genomic sequence portion complementary to a portion of the target genomic nucleic acid base sequence and a non-target-specific sequence that mismatches the target genomic nucleic acid base sequence;
(c) hybridizing the at least one labeled probe with the denatured target genomic nucleic acid base sequence at two or more sites on the target genomic nucleic acid base sequence to form a probe-target genomic nucleic acid base sequence hybrid including at least one mismatch;
(d) exposing the hybrid of step (c) to at least one repair enzyme selected from the group consisting of TDG and Mut Y, wherein said repair enzyme detects and cleaves the at least one mismatch to produce a cleaved nucleic acid base sequence; and
(e) detecting the cleaved nucleic acid base sequence of step (d).

The probe-target genomic nucleic acid base sequence hybrid of step (c) may comprise a T/G mismatch wherein a T is synthesized in the probe sequence to mismatch a G located in the genomic nucleic acid base sequence or a G is synthesized in the probe sequence to mismatch a T located in the genomic nucleic acid base sequence. Alternatively, the probe-target genomic nucleic acid base sequence hybrid of step (c) may comprise an A/G mismatch wherein an A is synthesized in the probe sequence to mismatch a G located in the genomic nucleic acid base sequence or a G is synthesized in the probe sequence to mismatch an A located in the genomic nucleic acid base sequence.

Optionally, the method further comprises, after step (e), the step of purifying the cleaved nucleic acid base sequence. The cleaved nucleic acid base sequence of step (d) may be labeled by hybridization to a biotin or fluorescent probe.

The target genomic nucleic acid base sequence is obtained from a source selected from the group consisting of a bacterium, fungus, virus, protozoan, plant, animal and human. In a preferred embodiment, the target genomic nucleic acid base sequence is obtained from a chicken.

The present invention relates to methods for identifying the presence of a Genomic DNA sequence utilizing the specificity of the base excision repair (BER) system enzymes, most preferably TDG and Mut Y. The target genomic DNA sample is renatured with a single stranded probe cleavable by a base excision repair enzyme. The probe is designed to be complementary or substantially complementary to a repetitive sequence that reoccurs often in the target DNA sample and that has a distinct affiliation to it. The probe includes an inserted point mutation relative to the target nucleic acid, whereby said mutation comprises an A/G point mutation to be detected and cleaved when Mut Y is used as the base excision repair enzyme, and whereby the mutation comprises a T/G point mutation to be detected and cleaved when TDG is used as the base excision repair enzyme. The probe preferably incorporates at least one label, preferably at least one resonance energy transfer donor moiety and at least one Black Hole Quencher (BHQ) moiety on opposite sides of the inserted point mutation no more than the quenching distance apart.

Specifically, a target genomic DNA sample is denatured and then renatured with a specially-designed single strand probe. The probe's sequence is constructed to be complementary to a repetitive sequence taken from a genomic database, which repetitive sequence reoccurs often in the target DNA and has a distinct affiliation to it. That is to say, the specific sequence chosen is affiliated with the selected genome and not another for the assay to work. For instance, where the assay is used to determine chick gender in ovo, the specific sequence chosen recognizes the female chicken genome but not the male genome. A point mutation is inserted at a desired location in the probe. The probe is labeled, preferably with a fluorescent label and also with a quencher within a quenching distance (FRET). A quencher decreases the fluorescence intensity of a given substance. A variety of processes can result in quenching, such as excited state reactions, energy transfer, complex formation and colissional quenching.

Several pairs of fluorescent labels and black hole quenchers (BHQ) may be constructed in the probe. Each pair of fluorescent label and the black hole quencher should be located on opposite sides of the inserted point mutation no more than the quenching distance apart. This distance is determined by the physical characteristics of the fluorescent label. Another option is for the fluorescent or the quencher to be attached directly to the inserted point mutation, while the other is located to the side of it no more then the quenching distance apart. The BHQ is preferred in this assay rather than just any acceptor moiety because the BHQ just absorbs fluorescence, but does not emit light itself, whereas other acceptors may be fluorescent labels themselves, but they also absorb and emit light and are therefore less suitable for this assay. A quencher that only absorbs but does not emit light by itself, i.e., is not a fluorescent label in itself, is thus preferred.

The mismatched nucleotide presented by the renaturation of the target DNA sample with the probe is cleaved with either TDG or Mut Y wherein the phosphate linkages at the resulting abasic sites are cleaved by treatment with alkali and/or heat treatment. For example:

If the probe is constructed so that it has a T instead of a C, thus creating a T/G point mutation to be detected and cleaved, then TDG is used to cleave the T/G.

If the probe is constructed so that it has an A instead of a C, thus creating an A/G point mutation to be detected and cleaved, then Mut Y is used to cleave the A/G.

The fluorescent signal that is formed when the probe is cleaved is then detected with a fluorescent reader.

The method of the invention is useful in an assay to determine if a chicken DNA sample is female or male chicken genome. Using chicken genome sequences obtainable from a database, a probe sequence is constructed complementary to a repetitive sequence taken from the database, which sequence reoccurs often in the target DNA and has a distinct affiliation to it. The probe is constructed having a complementary sequence, but with a point mutation.

The genomic sequence is selected from the group consisting of double stranded DNA and denatured, single stranded DNA or RNA.

The quencher moiety is preferably a Black Hole Quencher moiety. At least one resonance energy transfer donor moiety and at least one quencher moiety are situated on opposite sides of an inserted point mutation no more than the quenching distance apart. This means that there may be two pairs of fluorescent label (“F”) and quencher (“Q”) on the probe, each pair being on different sides of the mutation. This may be represented as:
_____F______F______T______Q______Q
______G______
   and would result in X2 signals, which would strengthen the signal.

Preferably, at least one resonance energy transfer donor moiety or the quencher moiety is attached directly to the inserted point mutation, and the other is located to the side of the inserted point mutation, no more than the quenching distance apart. This may be represented as:
______T/F______Q
______G______
   or, alternatively, as:
______T/Q______F
______G______
   It is critical that the single base pair mismatch is situated between the fluorescent moiety and the quencher moiety, otherwise the probe will not give a signal when cleaved.

### Genome Detection Assay

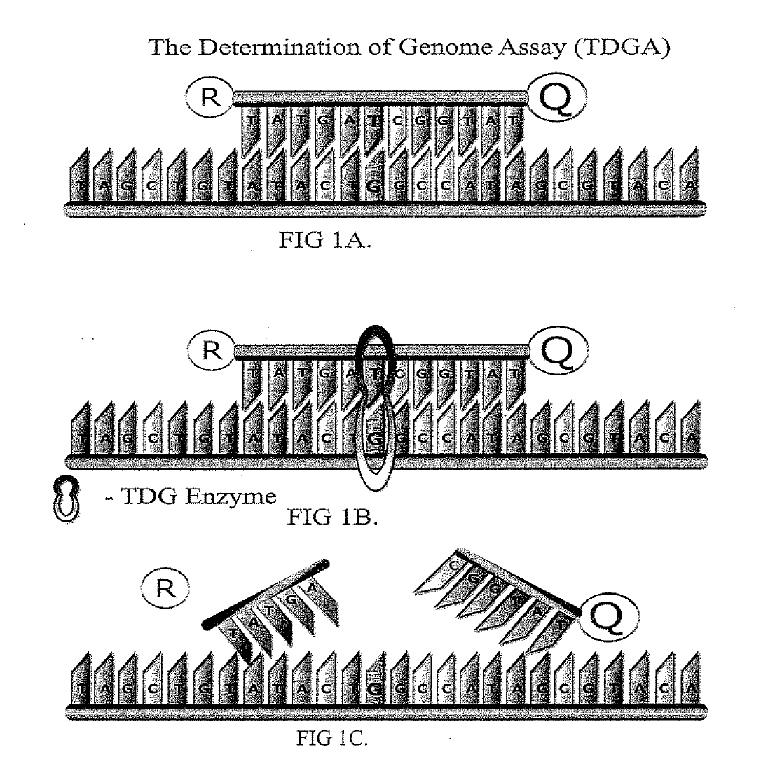

The principle underlying the genome detection assay lies in the fact that TDG functions solely on double stranded DNA. Genome specific repetitive sequences are employed to obviate the need for amplified target polynucleotide. Referring now to FIGS. 1A-1C, a probe is designed with a T in its sequence, to be complementary to the target non-amplified genomic DNA repetitive sequence except at the G that mismatches the T. The specially designed probe is hybridized to the target genomic DNA repetitive sequence, as shown in FIG. 1A. The probe is cleaved only if the specific T/G is formed. If an A is placed at the same point in the sequence, hybridization occurs and a T/A is formed, but is not susceptible to cleavage. As shown in FIG. 1B, the probe/genomic DNA complex is recognized by the mismatch repair enzyme TDG. In FIG. 1C, the probe is cleaved by TDG once it has identified the T/G, separating FRET and forming a signal which is then analyzed. In FIGS. 1A-1C, “Q” refers to the quencher and “R” refers to the reporter, a fluorescent label. The mismatch created by hybridization of the probe to the target DNA sequence acts as a switch which is then ‘turned on’ by TDG. As shown schematically in FIGS. 1A-C, the probe yields a fluorescence resonance energy transfer (FRET) signal: it is quenched when intact and fluorescent when cleaved by TDG. FIGS. 1A-1C demonstrate that a specific oligonucleotide probe can be cleaved by TDG when it has hybridized to its target gene, which has not been amplified by any technique. To give a strong signal, the sequence chosen is one which is frequently repeated in the female chicken genome, but rare in the male. Following this example, other species can be detected for their presence in non-lab settings according to their unique genomic sequence, using a single probe.

Five examples are presented below in which the TDG enzyme is shown to identify a mismatch. In Example I below, two PCR products of 152 bp are denatured and renatured to create a mismatch, which is identified by TDG. In Example II below, a PCR product of 164 bp is denatured and a fluorescent probe is introduced into the reaction creating a mismatch when hybridized to the PCR products. Part of the denatured PCR products are renatured with the probe and the T/G is recognized and cleaved by TDG. In Examples III and IV below, a FRET probe is tested and the mismatch then detected by running through a gel and also “as is” in solution. As the fluorescent moieties are bulky and are not the normal substrates for TDG, the object of the test was to determine if the efficiency of the TDG enzyme is compromised when the T from the T/G is situated between two close fluorescent nucleotides. When used in RTPCR, the efficiency of incorporation of such nucleotides by DNA polymerase tends to be much lower than the natural substrates. The results suggest that TDG can be used with FRET.

Having established that TDG can recognize and cleave a FRET probe and its activity is not affected by unevenness between the length of a probe and the target DNA, Example V below demonstrates that TDG can recognize a probe mismatched to non-amplified genomic DNA target. In Example V, genomic repetitive sequences were used instead of the PCR amplified products used in Examples I through IV. This is because with PCR amplified products, a sequence was chosen to mismatch the female sequence and match the male. Therefore, the female sequence gave rise to a cleaved band. PCR amplified sequences could not be used in Example V due to the fact that it is at a single and very specific gene location. The Chicken DNA has approximately one billion base pairs, which likely means that the probe will not specifically bind to a single site and the assay may not work. On a DNA as complex as the Chicken DNA it may bind to hundreds or thousands of sites depending on the sequence involved and the conditions used.

Furthermore, if the probe is to bind and be cleaved at the correct site, detecting it requires an amplification scheme due to the resolution limits of current fluorescent readers. As PCR is not suitable for non-lab settings, FISH results were examined for repetitive sequences (14, 15, 16, 17, 18) to provide an alternative to amplification by PCR. A repetitive sequence in the Chicken genome that would differentiate female from male was sought. Each time the probe binds to this repetitive sequence it will be cleaved in the female but not in the male and the intensity of the signal will depend on the number of the repetitive units in the genome. This number should be sufficient to overcome the resolution problem. A representative suitable candidate repetitive sequence was located.

As set forth below and as shown in the accompanying Figures, it is shown that TDG can recognize a probe on genomic DNA despite the size differences between the enzyme and probe to the target. Recognition occurs, TDG detects and cleaves the T/G in the probe/genomic DNA hybrid. However, partial and non-specific hybridization of the primer are possible, which may produce false positives in direct detection. This was shown to be overcome experimentally, where it was determined that the signal can be distinguished from the background, i.e., false positives.

### Definitions

Before addressing the features of various specific implementations of the present method in more detail, it is helpful to define certain terminology as used herein in the description and claims.

As used herein, “genomic DNA” is intended to include DNA from an organism's genome containing all coding (exon) and noncoding (intron and others) sequences in contrast to cDNA, which contains only coding sequences.

As used herein, the term “nucleic acid” is intended to include DNA molecules (e.g., genomic DNA) and RNA molecules (e.g., mRNA) and analogs of the DNA or RNA generated using nucleotide analogs sufficient for use as hybridization probes for the identification of a target nucleic acid. As referred to herein, nucleic acids that are “complementary” can be perfectly or imperfectly complementary, as long as the desired property resulting from the complementarity is not lost, e.g., ability to hybridize.

The nucleic acids of the present invention may be substantially isolated or alternatively unpurified. An “isolated” or “purified” nucleic acid is one that is substantially separated from other molecules that are present in the natural, source of the organism from which the nucleic acid is derived. Moreover, an “isolated” nucleic acid molecule can be substantially free of other cellular material, or culture medium when produced by recombinant techniques, or chemical precursors or other chemicals when chemically synthesized. (see, Sambrook et al. 1989, Molecular Cloning: A Laboratory Manual. 2nd, ed., Cold Spring Harbor Laboratory, Cold Spring Harbor Laboratory Press, Cold Spring Harbor, N.Y.).

A “hybridization probe” is a small molecule of DNA or RNA that is denatured (by heating) into single strands or synthesized as a single strand and then labeled, either radioactively or fluorescently or in some other known manner, and used to identify complementary nucleic acid sequences by hybridization.

Sequences which “hybridize” are those sequences binding under non-stringent conditions (e.g., 6.times SSC 50% formarnide at room temperature) and washed under conditions of low stringency (e.g., 2.times.SSC, room temperature, more preferably 2.times SSC, 42° C.) or conditions of higher stringency (eg. 2.times.SSC, 65° C.) (where NaCl, 0.015M sodium citrate, pH 7.2). Generally speaking, sequences that hybridize under conditions of high stringency are included within the scope of the invention, as are sequences which, but for the degeneracy of the code, would hybridize under high stringency conditions.

In the present description, the phrase “nucleic acid repair enzyme” denotes an enzyme that cleaves, at a point of mismatch, one strand of a duplex formed by oligonucleotide probe and target polynucleotide. Examples of nucleic acid repair enzymes that may be used in the methods of the invention are Thermostable Thymine-DNA Glycosylase (19) and Mut Y (20). The preferred TDG is E coli TDG (Trevigen, Inc., Gaithersburg, Md.), a thermostable enzyme known to remove thymidine from T:G mismatches. T:G mismatches form whenever a G:C and an A:T-containing fragment are cross-hybridized. Mut Y is also available from Trevigen, Inc.

### Analysis and Detection Methods

Various techniques are used to analyze DNA and RNA. For instance, Third Wave Technologies, Inc.'s Invader® Chemistry is composed of two simultaneous isothermal reactions. A primary reaction detects single-base changes, insertions, deletions, and changes in gene and chromosome number for genetics, chromosomal analysis, and infectious diseases. A second reaction is used for signal amplification. In the first reaction, two oligonucleotides, a primary probe and an Invader® oligo, associate with a DNA target to generate a one-base overlapping structure at the nucleotide being interrogated. If the variation or sequence in question is present, an overlapping structure is created with the mutant probe and the Invader® oligo on the target. Third Wave Technologies, Inc.'s Cleavase® enzymes specifically cleave the primary probes that form overlapping structures with the Invader® oligo, releasing the 5′ flaps plus one nucleotide. In the absence of the specific target, no flap is released. In the primary reaction, multiple probe molecules are cleaved per target molecule, and the signal generated from the cleaved 5′ flaps is amplified. The number of flaps released is relative to the amount of target in the sample.

Released flaps from the primary reaction serve as Invader® oligos in a second, simultaneous invasive cleavage reaction on a labeled, synthetic oligo, the fluorescence resonance energy transfer (FRET) probe. Cleavage of this FRET probe results in the generation of a fluorescent signal. Using two different 5′ flap sequences and their complimentary FRET oligos with non-overlapping fluorophores allows for two distinct sequences to be detected in a single well. Each released 5′ flap from the primary reaction cycles on and off the cleaved and uncleaved FRET probes, enabling cleavage of many FRET probes in the secondary reaction to further amplify the target-specific signal.

This technology is intended for both Genomic DNA targets and PCR products. There are other technologies that work with PCR to form a signal, such as the Taqman assay (21). This technique makes use of the 5′-exonuclease activity of a DNA polymerase to generate a signal by digesting a probe molecule to release a fluorescently labeled nucleotide. A target DNA containing a SNP is amplified in the presence of a probe molecule that hybridizes to the SNP site. The probe molecule contains both a fluorescent reporter-labeled nucleotide at the 5′-end and a quencher-labeled nucleotide at the 3′-end. The probe sequence is selected so that the nucleotide in the probe that aligns with the SNP site in the target DNA is as near as possible to the center of the probe to maximize the difference in melting temperature between the correct match probe and the mismatch probe. As the PCR reaction is conducted, the correct match probe hybridizes to the SNP site in the target DNA and is digested by the Taq polymerase used in the PCR assay. This digestion results in physically separating the fluorescent labeled nucleotide from the quencher with a concomitant increase in fluorescence. The mismatch probe does not remain hybridized during the elongation portion of the PCR reaction and is, therefore, not digested and the fluorescently labeled nucleotide remains quenched.

PCR may be carried out using materials and methods known to those of skill in the art. Analysis of amplification during real-time PCR is performed by detecting the fluorescence that is either directly or indirectly associated with the accumulation of the newly amplified DNA. The “Taqman” detection system uses a fluorescence resonance energy transfer (FRET) probe as a reporter system. A FRET probe is a short oligonucleotide that is complementary to one of the strands. The probe contains a “reporter” and a “quencher” fluorescent molecule at the 5′ and 3′ end of the probe, respectively. This probe is included in the real-time PCR reaction along with the required forward and reverse PCR primers. The quencher fluorochrome on the probe, because it is in such close proximity to the reporter, is able to quench the fluorescence of the reporter. As the Taq DNA polymerase enzyme replicates the new strand of DNA, the nuclease activity degrades the FRET probe at the 5′ end, which is bound to template DNA strand. This degradation releases the reporter fluorochrome from its proximity to the quencher, resulting in fluorescence of the reporter.

Various detection systems may be used depending on numerous factors including the desired specificity, assay development time, and cost per assay. Accumulation of fluorescent reporter molecules as a result of amplification of the target may be detected either indirectly by an appropriate optical sensing system such as the Taqman that detects the accumulation of fluorescence rather than the amplified DNA itself. Alternatively, it may be detected directly using a fluorescent DNA (SYBR green) that binds nonspecifically to double-stranded DNA, and the accumulation of the fluorescence bound to the amplified DNA target is measured.

Another direct approach is the “Molecular Beacon” technology that uses FRET-based fluorescent probes to bind the amplified DNA. In the unbound state, the quencher and reporter fluorochromes are maintained in close proximity via a hairpin loop designed into the sequence of the probe. Binding of the probe at a target sequence-specific region to its complementary strand on the amplified target DNA separates the two fluorochromes, reducing the FRET interference and allowing the reporter to fluoresce. Related systems using FRET-based PCR primers incorporated into the amplified DNA where the reporter and quencher fluorochromes are maintained in a hairpin loop structure via a sequence that is added to the 5′ end of one of the PCR primers have also been developed. In these systems, disruption of the hairpin loop structure during incorporation of the primer into the amplified DNA product results in loss of the FRET interference, leading to fluorescence of the reporter molecule.

The method of the invention differs significantly as the objective is to identify the presence of an entire genome rather than a specific gene. To identify Genomic DNA, the method of the invention uses repetitive sequences. Since the method of the invention does not seek to identify one SNP in a specific gene, but rather an entire genome, it does not require PCR or other amplification methods. Thus, the method of the invention can suffice with repetitive sequences to run the assay, without having to rely on PCR or other amplification methods.

The nucleic acids used in the methods of the invention may be labeled to facilitate detection in subsequent steps. The nucleic acids may be labeled, for example, by covalent attachment of one or more detectable groups. Any detectable group known to those skilled in the art may be used, for example, fluorescent groups, ligands and/or radioactive groups. Preferably, the probe is labeled with at least one molecule selected from the group consisting of a fluorescent label and black hole quencher, a fluorescent nucleotide, a fluorescent dye, biotin, derivative of biotin, radioactive molecule, fluorescent molecule, antibody, antibody fragment, hapten, carbohydrate, phosphorescent moiety, luminescent moiety, electrochemiluminescent moiety, chromatic moiety, nanostructure particle, and moiety having a detectable electron spin resonance, electrical capacitance, dielectric constant and electrical conductivity.

Detection of the labeled probe is by a method selected from the group consisting of PAGE, agarose gel, fluorescence reader detection system, sequencing, ELISA, mass spectrometry, fluorometry, hybridization, microarray, and Southern Blot.

### Preparation of Genomic DNA for Analysis

Nucleic acid molecules may be prepared for analysis using any technique known to those skilled in the art. Such nucleic acid extraction and preparation techniques may be found, for example, in Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), incorporated herein by reference.

When the nucleic acid of interest is present in a cell, it may be necessary to first prepare an extract of the cell and then perform further steps—i.e., differential precipitation, column chromatography, extraction with organic solvents and the like—in order to obtain a sufficiently pure preparation of nucleic acid. Extracts may be prepared using standard techniques in the art, for example, by chemical or mechanical lysis of the cell. Extracts then may be further treated, for example, by filtration and/or centrifugation and/or with chaotropic salts such as guanidinium isothiocyanate or urea or with organic solvents such as phenol and/or HCCl₃ to denature any contaminating or potentially interfering proteins. When chaotropic salts are used, it is usually desirable to remove the salts from the nucleic acid-containing sample. This can be accomplished using standard techniques in the art such as precipitation, filtration, size exclusion chromatography and the like.

In the present invention, denaturation and renaturation (annealing) of the target genomic DNA must be done. Denaturation is a process whereby a DNA molecule is converted from a two-stranded helical structure to a flexible, single-stranded structure, usually by applying heat. Renaturation is the reverse of the process, that is, cooling or otherwise reversing the process so that the single strands of DNA anneal to one another in a sequence specific manner to form double-stranded DNA.

### Enzymes

Base recognition/excision and repair techniques are described in, for example, S. Aziz, Annual Reviews of Biochemistry, 65:43-81 (1996); Kelley R., et al., Mutations, Plenum Press, New York (1996); P. Vaughan, Methods in Molecular Biology, vol. 152, DNA Repair Protocols, Prokaryotic Systems, Humana Press (2000); L. Stephen, et al., Annual Reviews of Biochemistry, 68:255-285 (1999); Au et al., Proc. Natl. Acad. Sci. USA, 86:8877-8881 (1989); D. Sheila and W. Scott, Nucleic Acids Research, 26(22): 5123-5133 (1998); L. A-Lien, et al., Proc. Natl. Acad. Sci. USA, 89:877-8783 (1992); and U.S. Pat. No. 6,008,031.

### TDG

Thymine-DNA glycosylase enzyme (TDG) recognizes T/G mismatches and cleaves the strand containing the T, correcting to C/G. At G/G mismatches TDG cleaves either strand, but not simultaneously, leaving a nicked DNA.

Since it was first described in 1989 (22, 23), TDG has been studied in various ways to understand the molecular mechanisms by which it achieves specificity for T/G mismatches and catalyzes accurate removal of the thymine base (T). The T is excised through N-glycosidic bond hydrolysis after deamination of 5-methylcytosine giving rise to T/G mismatches. Approaches for designing glycosylase inhibitors, such as transition state destabilization, have provided substantial insight into the structural basis for mismatch recognition and catalysis (24). This eventually led to the structure of TDG (25).

TDG is unique in that it recognizes its substrates solely in double strand DNA. It cleaves a normal, intact T, mismatched with G, unlike Uracil-DNA Glycosylase (UDG), which recognizes Uracil in single or double strand (23). This characteristic is central in developing the present assay to detect genomic DNA directly, for use in Biodiagnostic applications.

### Mut Y

Enzymatic systems capable of recognizing and correcting base pairing errors with a DNA strand have been demonstrated in bacteria, fungi and mammalian cells. Another example of a mismatch repair enzyme is Mut Y.

Mut Y is a glycosylase enzyme with a possible associated apurinic/apyrimidinic (AP) endonuclease (lyase) activity. Mut Y recognizes A/G mismatches in duplex DNA. DNA glycosylases cleave the glycosylic bond between the sugar and the base moieties of the DNA strand. Glycosylases are small enzymes (Mr=around 20,000 to 30,000) of narrow substrate specificity and have no cofactor requirement. DNA glycosylases cleave the glycosylic bond at the nitrogenous base, generating an apurinic/apyrimidinic (AP) (abasic) site. Once the base is cleaved by the glycosylase, AP endonucleases then cleave the phosphate backbone 3′ or 5′ to the abasic site. Enzymes having both glycosylase and AP endonuclease activities which generate 3′-OH and 5′-P (with or without the requirement for piperidine, in reactions that are known in the art) are even more preferred and can be used directly in the methods of the present invention.

### Preparation of a Sex-Specific Chicken Probe

In order to overcome the resolution problem of detection of the fluorescent probe, a repetitive sequence in the Chicken genome that differentiates female chickens from male chickens is selected. That is, the repetitive sequence is one that is repeated many times in the female chicken W chromosome, but is rare in the male chicken genome. Any of several specific repetitive DNA units in the chicken female W chromosome may be used. A representative repetitive DNA sequence which is a suitable candidate sequence is the 0.7 Xho I sequence that is repeated 20,000 times in the female chicken W chromosome and only 300 in the male genome (3). That is because a large fraction of one arm of the W chromosome is occupied by the XhoI family sequences. Other repetitive units also exist (15, 26, 27).

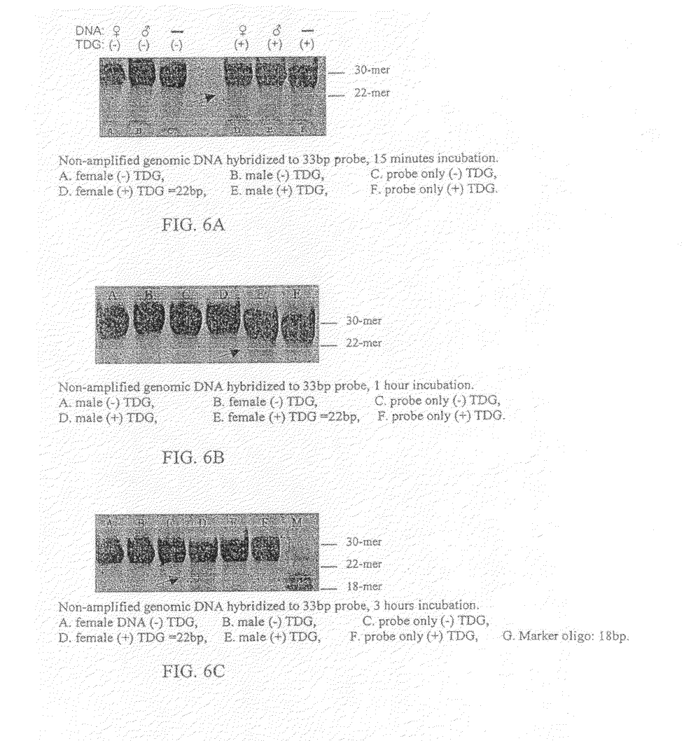

Each time the probe binds to this repetitive genomic DNA sequence, it will be cleaved by TDG in the female chicken but not in the male chicken. The intensity of the signal depends on the number of the repetitive units in the genome, which number should be sufficient to overcome the resolution problem. The specificity issue was shown not to be a concern. As shown in FIGS. 5 and 6, experimentally the operation is concentration dependent, and when the appropriate concentrations are present, then any “false mismatches” are background or non-existent with respect to the correct signal. That is, a correct signal to noise ratio is formed.

Concentrations of the target genomic DNA sequence, the probe and the enzyme are determined in order for this assay to work efficiently, as set forth in the accompanying Examples.

### Hybridizing the Primer to Genomic DNA

Non-amplified genomic chicken DNA, extracted from chicken female or male blood, was taken as is, denatured and then hybridized to a 30 bp probe, creating a mismatch if the DNA is female. The mismatch in the probe sequence is at point 22, so a 22 bp band is expected on denaturing PAGE (FIGS. 5, 6).

### EXAMPLE I

### TDG Identifies a Mismatch on a 152 bp PCR Amplified Product

A first test was conducted to verify the function of TDG on a mismatch located on 152 bp PCR amplified targets. PCR products containing a mismatch have been shown to be cleaved by TDG (28). Genes amplified by PCR from the chicken genome were used as the genomic DNA model. A candidate mismatch was chosen from the sex-linked marker on the female W sex chromosome, taken from the Chromobox-Helicase-DNA-binding gene. Two main gender determination tests were described based on the avian female W chromosome sex-linked gene, Chromobox-Helicase-DNA-binding (7). The female chicken has the CHDW and CHDZ alleles, while the male has only the CHDZ (x2). The 152 bp product sequence amplified from the CHD gene is similar between the Z and the W CHD genes except for 13 nucleotides that differ between the two (7). Nine of these are T/A to C/G transitions, which form T/G. The sequence is provided in reference (7) and the primers for this sequence are:

P2(7)5′TCTTGCATCGCTAAATCCTT3′,
PF152bp5′ATCAGCTTTAATGGGAGTGAAGGAAGACC3′

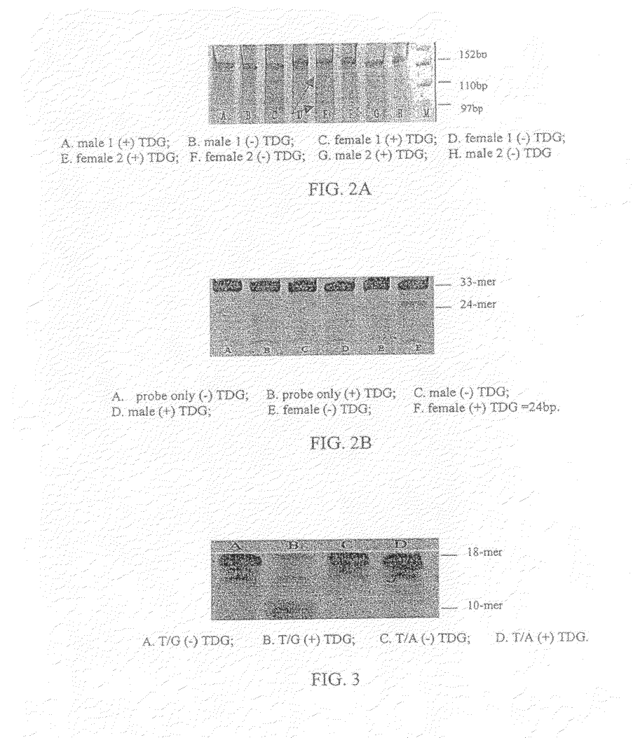

Two PCR products of 152 bp are denatured and renatured to create a T/G mismatch T/G mismatches are susceptible to TDG, which cleaves the T by hydrolysis of the N-glycosidic bond, thereby generating an abasic site. The phosphate linkages at the resulting abasic sites are cleaved by treatment with alkali and/or heat. This results in a single strand nick, cleaving the 152 bp band, and allowing the cleaved single strands to be detected on denaturing PAGE. FIG. 2A shows two females and two males treated (+) and not treated (−) with TDG. Two bands are shown in lanes C and E of FIG. 2A, which appear in the females treated with TDG because of the T/G formed by the difference between CHDW and CHDZ.

### EXAMPLE II

### TDG Identifies a Mismatch on a 164 bp PCR Amplified Product Where the Lengths of the Probe and the PCR Target Are Uneven

A second test was conducted on PCR amplified targets. It has been shown that TDG can recognize a short probe on a target of the same size (29). Other BER enzymes, such as MutY, were shown to do this as well (30,31). The object of this test is to show that TDG will recognize the TIG that is formed if a probe is hybridized to the PCR target, even when their lengths are uneven, i.e. 33 bp to 164 bp. A 5′fluorescently labeled 33 bp probe (FA33F) was synthesized to complement a section from the 164 bp PCR product (152 bp+12 bp), except for a mismatch that would differentiate CHDZ from CHDW. The probe contained a T matched to A on CHDZ and mismatched to G on CHDW at the same location in the sequence. The probe competes with the original 164 bp PCR product. Six possibilities now exist in the solution: C/G, C/A formed from the PCR product alone and T/G or T/A formed from either the PCR product alone or the probe/PCR hybrid.

As shown in FIG. 2B, results showed that for the 164 bp PCR product amplified from CHD gene hybridized to 33 bp probe, the T at location 24 bp on the probe in the T/G probe/PCR complex is successfully recognized and cleaved by TDG. In lane F, Female treated with TDG=24 bp, CHDW was recognized, while the T/A probe/PCR hybrid at CHDZ remained intact.

### EXAMPLE III

### TDG Identifies A Mismatch on a 18 bp Synthetic Primer Hybridized to an 18 pb FRET Dual Label Probe Using PAGE Analysis

The use of PAGE allowed visualization of the assay. However, this type of analysis is laborious and only suitable under laboratory conditions. For the assay to function under demanding non-lab conditions, with an immense number of samples, it must be fast, allow parallel analysis, and withstand other restrictions.

If a fluorescence resonance energy transfer (FRET) dual label probe is used, it is possible to translate PAGE analysis to analysis in solution. This requires that the TDG enzyme cleave a TIG in the presence of the two fluorophores, when only 18 base pairs separate the two. TDG has been shown to protect a ˜20 bp oligonucleotide from DNase I cleavage (24). An experiment was run to determine whether, with such a short distance between them, there would be steric interference of the bulky fluorescent moieties that would disrupt the identification and specificity of TDG.

An 18 bp probe was labeled at the 5′ prime with FAM (6-carboxyfluorescein) and with TAMRA (6-carboxyltetramethylrhodmine) at the 3′ prime and contained a T at location 10 bp in the sequence. A matched or mismatched target of the same length was hybridized to the probe and once again cleaved with TDG and run through denaturing PAGE, as shown in FIG. 3, where Lane A=T/G (−) TDG; Lane B=T/G (+) TDG; Lane C=T/A (−) TDG; and Lane D=T/A (+) TDG, where “+” indicates treated and “−” indicates untreated.

### EXAMPLE IV

### TDG Identifies a Mismatch on a 18 bp Synthetic Primer Hybridized to an 18 pb FRET Dual Label Probe Using Laser Detection Analysis

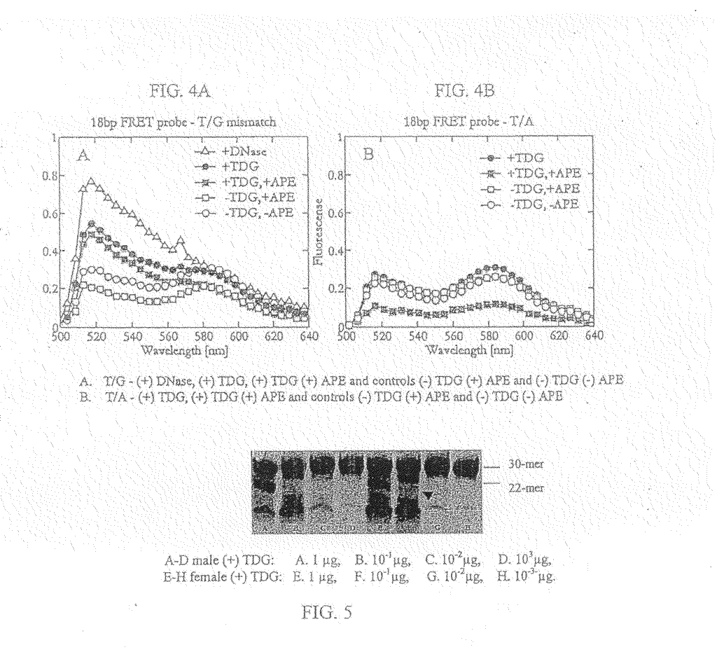

The experiment was repeated under slightly different conditions. Instead of running the sample through a gel, it was analyzed with a laser detection system. Results were similar to gel (FIG. 3) and showed that, when an 18 bp FRET probe is mismatched (T/G) to an 18 bp target, the double strand is cleaved. Once the probe is cleaved and denatured from its target, the reporter fluorochrome is released from its proximity to the quencher, resulting in fluorescence of the reporter at 520 nm (FIG. 4A). When an 18 bp FRET probe is matched (T/A) to an 18 bp target, with TDG, the fluorescence level is equal to T/A or T/G, without TDG (controls), suggesting that the double strand was not cleaved (FIG. 4B).

TDG is able to cleave the DNA at the site of the mismatch, between two closely situated fluorophores and without mistakenly identifying the fluorophores as somehow mismatched bases. Furthermore, we tested if TDG plus alkali buffer alone can cleave the double strand or if Apurinic/apyrimidinic endonuclease (APE) would be required, in order to cleave the phosphate linkage at the resulting abasic site. Results shown in FIGS. 4A and 4B demonstrate that cleavage with TDG plus alkali buffer is compatible with or almost the same as TDG plus APE. APE alone, without TDG, shows fluorescence level equal to T/A or T/G, without TDG (controls). Therefore, TDG plus alkali buffer alone is sufficient for this assay (FIGS. 4A-4B). The graphs are representative of one experiment out of a series with repeated results. The experiment was repeating using used MutY (a glycosylase with Apurinic/apyrimidinic endonuclease activity) instead of TDG (the mismatch was changed to A/G) and results were the same (data not shown).

### EXAMPLE V

### TDG Identifies a Mismatch on Non-Amplified Genomic DNA Target

FIG. 5 is a gel that shows that when non-amplified genomic DNA hybridized to a 30 bp probe, the ratio between the concentration of the non-amplified genomic DNA and the probe is critical to the success of the assay. Lanes A-D are male chicken genomes treated with the following amounts of TDG: A. 1 μg; B. 10⁻¹ μg; C. 10⁻² μg; D. 10³ μg. Lanes E-H are female chicken genomes treated with the following amounts of TDG: E. 1 μg; F. 10⁻¹ μg; G. 10⁻² μg; H. 10⁻³⁻μg. The results demonstrate that 1 μg of the male sample shows the same band at 22 bp as the female, but in one tenth of that the female band is more apparent than the male's. This result is to be expected because when there is excess DNA in solution it is more likely that non-specific hybridization will occur, forming false positive mismatches and thus a false positive signal. Other bands that appear correlate to T's in the probe sequence which give rise to false positives.

In view of the results shown in FIG. 5, the test was repeated using the same probe as in FIG. 5, but with 50 ng of genomic chicken DNA. The test was run for 15 min (FIG. 6A) 1 hour (FIG. 6B) and 3 hours (FIG. 6C). The results, shown in FIGS. 6A-6C, demonstrate that there is a sex-specific mismatching base at position 22, and that the heteroduplex can be cleaved by TDG to yield a 22 nucleotide band. FIG. 6A confirms that this assay is applicable and furthermore, can be done within a fifteen-minute incubation time. FIGS. 6B and 6C show one and three hours incubation correspondingly.

Thus, FIGS. 6A-6C demonstrate that a genome sequence can be directly detected by TDG. The combination of TDG with the probe favors detection of a specific sequence in genomic DNA over a partial or non-specific sequence, which would form false positive results. The fact that a band appears in the female sample, but not in the male or control samples, indicates that correct hybridization occurred, followed by correct cleavage. This was shown to be concentration dependent. The results demonstrate that direct detection of repetitive sequences is possible. It is likely that further improvements can be obtained that will allow direct detection of single sequences. This would require development of an appropriate amplification system.

### EXAMPLE VI

Using the method of the invention, it is possible to take a 5-50 micro-liter sample of blood from a chicken egg and determine, within a 15-minute reaction time, if it is a female or male chicken. This method may be used at the hatchery to segregate the eggs before they hatch.

An assay is performed on Genomic DNA extracted from 50 microL of chicken blood. While the experiment was conducted using a specially designed probe prepared from a repetitive sequence in accordance with the method of the invention, there are any number of suitable probes that may be constructed using other repetitive sequences that may be used to identify the Chicken genome.

The results of a PAGE gel with fluorescent imaging using the chicken genomic DNA is shown in FIG. 6A, 6B, and 6C. The fluorescent band shown by the arrow indicates the existence of the female chromosome W, as detected by the specially-designed probe cleaved with TDG. Lanes from the left: Without TDG enzyme- female, male and probe alone; With TDG enzyme—female, male and probe alone. “M” in the lower right of FIG. 2 indicates the marker DNA.

### EXAMPLE VII

### Specific Genome Sequence Isolation Assay

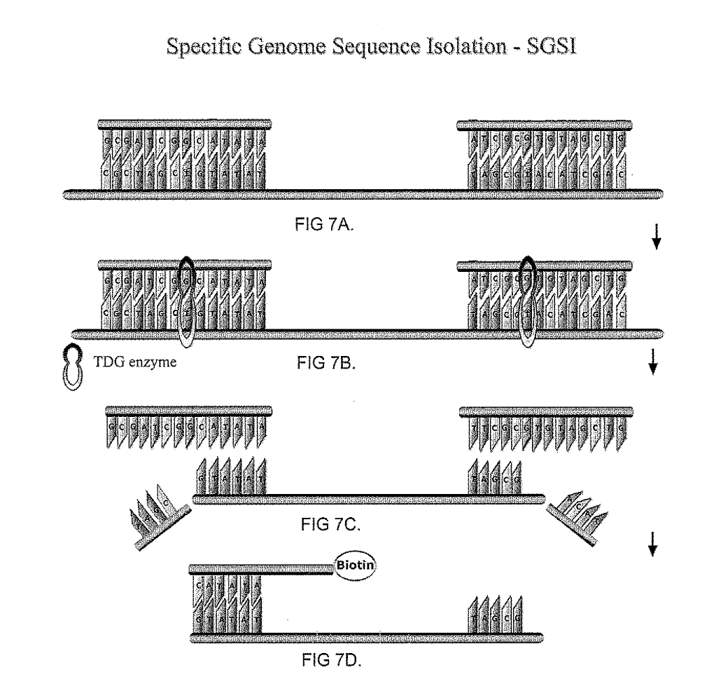

FIGS. 7A-7D illustrates a specific genome sequence isolation (SGSI) assay. In FIG. 7A, two specially-designed probes are hybridized to the denatured target genomic DNA sequences at two or more sites on the target genomic nucleic acid base sequence. Each probe, which is preferably labeled, comprises a single-stranded nucleic acid having a target-specific genomic sequence portion complementary to a portion of the target genomic nucleic acid base sequence and a non-target-specific sequence that mismatches the target genomic nucleic acid base sequence. In FIG. 7B, the probe/target genomic DNA hybrids including at least one mismatch are detected by the mismatch repair enzyme TDG. In FIG. 7C, the specific genomic DNA sequence is cleaved by TDG having identified the thymines in the target mismatches. Finally, in FIG. 7D, a biotinilated probe is attached to the cleaved sequence and subsequently isolated through avidin-magnet purification to detect the cleaved nucleic acid base sequence.

### Applications

Due to its reduced detection times and cost effectiveness, the invention will find use in environmental monitoring and nucleic acid diagnostics. It is contemplated that devices will be developed that will allow rapid FRET analysis and the method of the invention to be performed at the point the sample is collected rather than in a distant laboratory requiring specialized laboratory services.

Although the method is described using Chicken DNA as a model, the method may be used to identify other species specific DNA under non-lab settings. For instance, the method of the invention may be used to identify Anthrax, for example, by integrating the assay in an apparatus, similar to a wall-mounted smoke detector, capable of detecting the presence of a bacterium or virus DNA. It may find application in a Doctor's office to determine the gender of a fetus a woman is carrying after 8 weeks of pregnancy. It can conceivably be used to check for various viruses at airports or other places to ensure that suspicious subjects are not spreading disease. In these applications, a subject's blood sample or DNA sample is taken and analyzed through the assay that is integrated into a small device, with detection possible in small, disposable systems that produce a signal to indicate positive results or that are readable through attachments to a personal computer. This technology would enable point-of-care for nucleic acids from bacterial or viral pathogens. The device would incorporate nucleic acid extraction capability, temperature changing-capability to permit denaturing and renaturing of DNA, and FRET. It is envisioned that the DNA would be extracted, but not necessarily ‘cleaned’. The DNA is denatured and renatured with the FRET probe, the assay is preformed and the probe is detected. In the case of bacteria or virus or allantoic fluid from the egg, it is envisioned that a DNA sample may be obtained without cleaning the DNA, and the assay may be preformed directly after the DNA is heated to be denatured. Therefore, a small, portable detector device may be suitable as no DNA cleaning is required. Fluorescence labels may be taken from the red light range, so that inexpensive diodes may be used as the ‘laser’. In the event a blood sample is analyzed in the assay of the invention, such as when blood is obtained from a pregnant female, then the preliminary steps of DNA extraction and cleaning may be required. Other applications of the methods of the present invention should occur to those skilled in the art.

The invention further provides kits for determining the gender of a bird or egg of unknown gender comprising (a) a sample genomic nucleic acid base sequence from a bird or target egg of interest of unknown gender; (b) at least one hybridization probe comprising a single-stranded nucleic acid having a target-specific genomic sequence portion complementary to a portion of the target genomic nucleic acid base sequence and a non-target-specific sequence that mismatches the target genomic nucleic acid base sequence or said at least one probe joined to a label; (c) at least one repair enzyme selected from the group consisting of TDG and Mut Y; and (d) instructions for determining the gender of the bird or egg. The sample genomic nucleic acid base sequence may derive from a target egg of interest of unknown gender or, alternatively, from a newly hatched or adult bird, such as in the case of sexing individual birds such as parrots. The DNA from the egg may come directly from the chick or from cells in the fluid or from blood on the egg shell.

Methods:

Cleavage Assays:

The standard reaction mixture: 10 μl contained 1×REC Buffer (Trevigen, Inc.) 10 mM HEPES-KOH (pH 7.4), 100 mM KCl, and 10 mM EDTA. 5 units of TDG (Trevigen, Inc.) 18 bp FRET probe detected by gel; 0.1 picomol labeled probe Fam and 0.1 picomol of its mismatched sequence Fami or its matched sequence Oli were added to the standard reaction mixture. Fam/Fami or Fam/Oli were incubated at 37° C. for 2 hr and the reaction was then stopped as above and loaded onto a 20% denaturing polyacrylamide gel for ½ hr at 160V. The bands were visualized on the Typhoon™ 8600 Variable Mode Imager (GE Healthcare Life Sciences).

Fam*5′(6FAM)ACTGCGTCTT*CCTTCACT (TAMRA)3′
Fami5′AGTGAAGGGAGACGCAGT3′
Oli5′AGTGAAGGAAGACGCAGT3′
Famc5′ACTGCGTCTTCCTTCACT3′

18 bp FRET probe detected in solution; another step was added to the above stated reaction; after a 2 hr incubation at 37° C., 1 picomol primer was added to the reaction, which is identical to the FRET probe, but is not labeled (Fame). The double strand was then heated at 94° C. and then incubated at 37° C. for 1 min. The Fame unlabeled primer competed for the mismatched or matched primers, leaving the FRET probe cleaved and free and therefore readable by the laser detection system as follows.

The Laser Optical Detection System

FRET probe detection in solution was accomplished using an Argon laser (488 nm, 261B power supply, Spectra-Physics Lasers, Mountain View, Calif.) and a fiber optic spectrometer (USB2000, Ocean Optics, Dunedin, Fla.). The excitation laser beam was directed onto a 0.2 ml PCR tube using a 506 nm long-pass filter (LPF-506-25.2mm×35.6 mm-HC, CVI Laser, Albukuerque, N. Mex.). The fluorescence was collected using a plano-convex lens (focal length, 38.1 mm), transmitted through a bandpass filter specific for 6-FAM (F40-536.0-25 mm-HC, CVI Laser, Albukuerque, N. Mex.) and detected by the fiber optic spectrometer.

Genome Detection Assay'—TDGA

To determine the female or male origin of the sample, final experiments were done as follows: 5×10⁻² μg of genomic DNA either from female or male were added to the standard reaction mixture (7.5 units TDG), heated at 94° C. for 10 min and held at 60° C. for 15 min, 1 hr or 3 hr and stopped by adding 10 μl TBE-Urea Sample Buffer (Bio-Rad) and heated at 94° C. for 10 min. The sample was loaded onto a 20% denaturing polyacrylamide gel for ½ hr at 160V. The bands were visualized on the Typhoon™ 8600 Variable Mode Imager (GE Healthcare Life Sciences).

Genomic DNA Extraction

Genomic DNA was extracted from 5 and 50 μl fresh blood of mature chickens using GFX Genomic Blood Purification kit (Amersham. Biosciences). The purified Genomic DNA was used as a template for PCR reaction.

PCR primer sequences were taken from the CHD genes as follows:

P2(7)5′TCTTGCATCGCTAAATCCTT3′,
PF152bp5′ATCAGCTTTAATGGGAGTGAAGGAAGACC3′

PCR was performed on an MJ research PTC 200 Thermal Cycler, an initial denaturing step at 94° C. for 5 min was followed by 35 cycles of 94° C. for 30 sec, 48° C. for 30 sec and 68° C. for 30 sec. A final run of 72° C. for 10 min completed the program.

PCR samples: PCR samples were taken as a substrate for the cleavage reaction. 10-40 ng/μl of PCR samples from male and female chickens were added to the standard reaction, heated at 94° C. for 10 min and then incubated at 37° C. for 2 hr. The reaction was stopped by adding 10 μl TBE-Urea Sample Buffer (Bio-Rad) and heated at 94° C. for 10 min. The sample was loaded onto a 20% denaturing polyacrylamide gel for 1.5 hr at 300V.

The bands were visualized by DNA Silver Staining Kit (Amersham Biosciences), according to the manufacturer's instructions.

PCR probe reaction: 10-40 ng/μl PCR samples plus 0.1 picomol FA33F probe (Danyel Biotech, Ltd., Rehovot, Israel) were added to the standard reaction, heated at 94° C. for 10 min and then incubated at 37° C. for 2 hr.

FA33F 5′(Cy3)ATATCTTCTGCTCCTACTGCGTCTT*CCTTCACT3′.

The reaction was stopped as above and then loaded onto a 20% denaturing polyacrylamide gel for ½ hr at 160V. The fluorescent bands were visualized on the Typhoon™ 8600 Variable Mode Imager (GE Healthcare Life Sciences). The primers used to generate the PCR products for this assay were P2 (as above) and PH 64 bp5′CTGAAATTCCAGATCAGC3′.

18 bp FRET probe used with MutY; a primer was synthesized to create an A/G mismatch -Fe5′AGTGAAGGAAGACGCAGG3′. 10 μl contained 1×REC Buffer (Trevigen Inc.) 10 mM HEPES-KOH (pH 7.4), 100 mM KCl, and 10 mM EDTA. 1 unit of MutY (Trevigen Inc.). 0.1 picomol of each Fam/Fe or Fam/Oli were added and incubated at 37° C. for 2 hr. Then 1 picomol of the unlabeled Fe primer (Danyel Biotech, Ltd., Rehovot, Israel), was added and the reaction was heated at 94° C. and then incubated at 37° C. for 1 min and then detected as above.

Genomic samples: 1, 10⁻¹, 10⁻², 10⁻³ μg were taken accordingly from male or female chicken as stated above. DNA sample was determined by PicoGreen dsDNA quantitation (Invitrogen), according to the manufacturer's instructions. Samples were added to the standard reaction mixture (with 7.5 units instead of 5), heated at 94° C. for 10 min and then held at 60° C. for 3 hr. It was stopped as above and then samples were loaded onto a 20% denaturing polyacrylamide gel for ½ hr at 160V. The bands were visualized on the Typhoon™ 8600 Variable Mode Imager (GE Healthcare Life Sciences).

While the present invention has been described with reference to specific applications or embodiments, it should be understood by those skilled in the art that various changes may be made and equivalents may be substituted without departing from the true spirit and scope of the invention. In addition, many modifications may be made to adapt a particular situation, material, composition of matter, process or assay to the objective, spirit and scope of the present invention. All such modifications are intended to be within the scope of the invention.

All references cited herein are to aid in the understanding of the invention, and are incorporated in their entireties for all purposes.

### REFERENCES

1. www.factoryfarming.com
2. D. Bell, Egg economics update report (May 2005).
3. Tone, M., Sakaki, Y., Hashiguchi, T. & Mizuno, S. Genus specificity and extensive methylation of the W chromosome-specific repetitive DNA sequences from the domestic fowl, Gallus gallus domesticus, Chromosoma, 89 (3), 228-237 (1984).
4. Marshall Graves, J. A. & Shetty, S. Sex from W to Z: Evolution of Vertebrate Sex Chromosomes and Sex Determining Genes, Journal of Experimental Zoology. 290, 449-462.
5. Hori, T., Asakawa, S., itoh, Y., Shimizu, N. & Mizuno, S. Wpkci, Encoding an altered form of PKCI, is conserved widely on the avian W chromosome and expressed in early female embryos: implication of it role in female sex determination, Molecular Biology of the Cell, 11, 3645-3660 (2000).
6. Fridolfsson, A. K. et al. Evolution of the avian sex chromosomes from an ancestral pair of autosomes, Proc. Natl. Acad. Sci. USA 95, 8147-8152 (1998), Genetics.
7. Griffiths, R., Double M. C., Orr K. & Dawson R. J. G. A DNA test to sex most birds, Molecular Ecology. 7, 1071-1075 (1998).
8. Archawaranon, M., Rapid sexing Hill Mynah Gracula religiosa by sex chromosomes, Biotechnology, 3(2):160-164 (2004).
9. Manuel, R. C., Czerwinski, E. W. & Lloyd, R. S. The Identification of the structural and functional domains of MutY an Escherichia coli DNA mismatch repair enzyme, Journal of Biological Chemistry. 271 (27), 16218-16226 (1996).
10. Chepanoske, C. L., Porello, S. L., Fujiwara, T., Sugiyama, H. & David, S. S. Substrate recognition by Escherichia coli MutY using substrate analogs, Nucleic Acid Research. 27 (15), 3197-3204 (1999).
11. Rosi, L. N. & Mirkin, A. C. Nanostructures in Biodiagnostics, Chem. Rev. 105, 1547-1562 (2005).
12. MIDAScan™ AT Mutation Detection Kit, Trevigen, Inc.
13. Porter, A. C. G., Two Hands Make Light Work of Gene Modification, Rejuvenation Research, 8 (4) Volume 8, 211-215 (2005).
14. Umov, F. D. et al., Highly efficient endogenous human gene correction using designed zinc-finger nucleases, Nature, 435|2 646-651 (2005).
15. Saitoh, Y. & Mizuno, S. Distribution of Xho I and EcoR I family repetitive DNA sequences into separate domains in the chicken W chromosome, Chromosoma, 101 (8), 474-477 (1992).
16. Chen, Z. Q., Ritzel, R. G., Lin, C. C. & Hodgetts, R. B. Sequence conservation in avian CR1: An interspersed repetitive DNA family evolving under functional constraints, Proc. Natl. Acad. Sci. USA 88 (18), 5814-5818 (1991), Evolution.
17. Tone, M., Nakano, N., Takao, E., Narisawa, S. & Mizuno, S. Demonstration of W chromosome-specific repetitive DNA sequences in the domestic fowl, Gallus g. domesticus, Chromosoma, 86 (4), 551-569 (1982).
18. Solovei, I., Macgregor, H. & Gaginskaya, E. Single stranded nucleic acid binding structures on chicken lampbrush chromosomes, Journal of cell science, 108, 1391-1396 (1995).
19. Begley, T. J., and R. P. C. Cunningham.1999. Methanobacterium thermoformicicum thymine DNA mismatch glycosylase; conversion of an N-glycosylase to an AP lyase. Protein Engineering 12:333-340.
20. Wu et al., Proc. Nat'l Acad. Sci. USA 89: 8779-83 (1992).

21. Arnold, et al., BioTechniques 25(1):98-106 (1998).
22. Wiebauer, K. & Jiricny, J. In vitro correction of GT mispairs to GC pairs in nuclear extracts from human cells, Nature (London) 339, 234-236 (1989).
23. Wiebauer, K. & Jiricny, J. Mismatch-specific thymine DNA glycosylase and DNA polymerase P8 mediate the correction of G T mispairs in nuclear extracts from human cells, Proc. Natl. Acad. Sci. USA 87, 5842-5845 (1990).
24. Scharer, O. D., Kawate, T., Gallinari, P., Jiricny, J. & Verdine, G. M. Investigation of the mechanisns of DNA binding of the human G/T glycosylase using designed inhibitors, Proc. Natl. Acad. Sci. USA 94, 4878-4883 (1997).
25. Mol, C. D., Arvai, A. S., Begley, T. J., Cunnungham, R. P. & Tainer, J. A. Structure and activity of a thermostable thymine-DNA glycosylase: evidence for base twisiting to remove mismatched normal DNA bases, J. Mol. Biol. 315(3): 373-384 (2002).
26. Wicker, T. et al. The repetitive landscape of the chicken genome, Genome Res. 15, 126-136 (2005).
27. Saitoh, Y. & Mizuno, S. Molecular and cytological characterization of SspI-family repetitive sequence on the chicken W chromosome, Chromosome Research. 10, 499-511 (2002).
28. Hsu, I. C., Yang Q., Kahng M. W., Xu J. F. Detection of DNA point mutations with DNA mismatch repair enzymes, Carcinogenesis 15, 1657-1662 (1994).
29. Ullah, S. & Rufus, S. D. DNA- substrate sequence specificity of human GT mismatch repair activity, Nucleic Acid Research. 21 (5), 1281-1287 (1993).
30. Tsai-Wu, J. J., Liu, H. F. & Lu, A. L. Escherichia coli MutY protein has both N-glycosylase and apurinic/apyrimidinic endonuclease activities on AC and AG mispairs, Proc. Natl. Acad. Sci. USA 89 (18), 8779-8783 (1992).
31. Williams, S. D. & David, S. S., Evidence that MutY is a monofunctional glycosylase capable of forming a covalent Schiff base intermediate with substrate DNA, Nucleic Acid Research. 26 (22), 5123-5133 (1998).
Sundstrom, H., Webster, M. T. & Ellegren, H. Reduced variation on the chicken Z chromosome, Genetics, 167, 377-385 (2004).
Holmes, J. JR., Clark, S. & Modrich, P. Strand-specific mismatch corrections in nuclear extracts of human and Drosophila melanogaster cell lines, Proc. Natl. Acad. Sci. USA 87, 5837-5841 (1990), Biochemistry.

I claim:

1. A method for detecting and cleaving a target non-amplified genomic nucleic acid sequence at two or more genomic nucleic acid sequence sites simultaneously, using a mismatch repair enzyme selected from the group consisting of TDG and Mut Y, comprising, for each genomic nucleic acid sequence site to be detected and cleaved, the steps of:
(a) denaturing the target genomic nucleic acid base sequence;
(b) preparing at least one labeled probe comprising a single-stranded nucleic acid having a target-specific genomic sequence portion complementary to a portion of the target genomic nucleic acid base sequence and a non-target-specific sequence that mismatches the target genomic nucleic acid base sequence;
(c) hybridizing said at least one labeled probe with said denatured target genomic nucleic acid base sequence at two or more sites on the target genomic nucleic acid base sequence to form a probe-target genomic nucleic acid base sequence hybrid including at least one mismatch;
(d) exposing the hybrid of step (c) to at least one repair enzyme selected from the group consisting of TDG and Mut Y, wherein said repair enzyme detects and cleaves said at least one mismatch to produce a cleaved nucleic acid base sequence; and
(e) detecting the cleaved nucleic acid base sequence of step (d).

2. The method of claim 1 wherein the probe-target genomic nucleic acid base sequence hybrid of step (c) comprises a TIG mismatch wherein a T is synthesized in said probe sequence to mismatch a G located in the genomic nucleic acid base sequence.

3. The method of claim 1 wherein the probe-target genomic nucleic acid base sequence hybrid of step (c) comprises a TIG mismatch wherein a G is synthesized in said probe sequence to mismatch a T located in the genomic nucleic acid base sequence.

4. The method of claim 1 wherein the probe-target genomic nucleic acid base sequence hybrid of step (c) comprises an AIG mismatch wherein an A is synthesized in said probe sequence to mismatch a G located in the genomic nucleic acid base sequence.

5. The method of claim 1 wherein the probe-target genomic nucleic acid base sequence hybrid of step (c) comprises an AIG mismatch wherein a G is synthesized in said probe sequence to mismatch an A located in the genomic nucleic acid base sequence.

6. The method of claim 1 wherein, after step (e), the step of purifying the cleaved nucleic acid base sequence.

7. The method of claim 1 wherein said probe is labeled with at least one molecule selected from the group consisting of a fluorescent label and black hole quencher, a fluorescent nucleotide, a fluorescent dye, biotin, derivative of biotin, radioactive molecule, fluorescent molecule, antibody, antibody fragment, hapten, carbohydrate, phosphorescent moiety, luminescent moiety, electrochemiluminescent moiety, chromatic moiety, nanostructure particle, and moiety having a detectable electron spin resonance, electrical capacitance, dielectric constant and electrical conductivity.

8. The method of claim 1 wherein detection of the labeled probe in step (e) is by a method selected from the group consisting of PAGE, agarose gel, fluorescence reader detection system, sequencing, ELISA, mass spectrometry, fluorometry, hybridization, microarray, and Southern Blot

9. The method of claim 1 wherein the cleaved nucleic acid base sequence of step (d) is labeled by hybridization to a biotin or fluorescent probe.

10. The method of claim 1, wherein said target genomic nucleic acid base sequence is obtained from a source selected from the group consisting of a bacterium, fungus, virus, protozoan, plant, animal and human.

11. The method of claim 1, wherein said target genomic nucleic acid base sequence is obtained from a chicken.

12. A kit for determining the gender of a bird or egg of unknown gender, comprising:
(a) a sample genomic nucleic acid base sequence from a bird or target egg of interest of unknown gender
(b) at least one hybridization probe comprising a single-stranded nucleic acid having a target-specific genomic sequence portion complementary to a portion of the target genomic nucleic acid base sequence and a non-target-specific sequence that mismatches the target genomic nucleic acid base sequence or said at least one probe joined to a label;
(c) at least one repair enzyme selected from the group consisting of TOG and Mut Y; and
(d) instructions for determining the gender of the bird or egg.


SEQUENCE LISTING (8 sequences)

SEQ ID NO: 1 - DNA, 18 bp, Gallus gallus
actgcgtcttccttcact

SEQ ID NO: 2 - DNA, 18 bp, Gallus gallus
agtgaaggaagacgcagt

SEQ ID NO: 3 - DNA, 18 bp, Gallus gallus
agtgaagggagacgcagt

SEQ ID NO: 4 - DNA, 18 bp, Gallus gallus
agtgaaggaagacgcagg

SEQ ID NO: 5 - DNA, 33 bp, Gallus gallus
atatcttctgctcctactgcgtcttccttcact

SEQ ID NO: 6 - DNA, 30 bp, Gallus gallus
actcctctcctctcctctcctctcctttcc

SEQ ID NO: 7 - DNA, 18 bp, Gallus gallus
ctgaaattccagatcagc

SEQ ID NO: 8 - DNA, 29 bp, Gallus gallus
atcagctttaatgggagtgaaggaagacc